(19) United States
(12) Patent Application Publication
(10) Pub. No.: US 2004/0048369 A1
(43) Pub. Date: Mar. 11, 2004

(54) Control of inward-rectifier K+ ion channels

(76) Inventor: Zhe Lu, Wynnewood, PA (US)

Correspondence Address: Evelyn H. McConathy, Esquire, Dilworth Paxson LLP, 3200 Mellon Bank Center, 1735 Market Street, Philadelphia, PA, 19103, US

(21) Appl. No.: 10/655,190

(22) Filed: Sep. 4, 2003

Related U.S. Application Data
(60) Provisional application No. 60/408,398, filed on Sep. 4, 2002.

(51) Int. Cl. A61K 31/198; C12N 5/06

(52) U.S. Cl. 435/325; 514/566

(57) ABSTRACT

Provided are methods for controlling activity of an inward rectifier K⁺ (Kir) ion channel in a cell or cell membrane by controlling an amount of one or more chemical contaminant(s) of an organic pH buffer or metal chelator in intracellular solution. Also provided are methods for treating a patient requiring enhancement of cardiac contractility, wherein one such method comprises administering to the patient an amount of piperazine, or a derivative thereof, sufficient to reduce or block the activity of at least one inward rectifier ion channel, thereby prolonging cardiac action potential, which causes voltage-gated Ca⁺⁺ channels to remain open for a period longer than the voltage-gated Ca⁺⁺ channels would be open absent such addition of piperazine; thereby enhancing Ca⁺⁺ entry into myocytes of the patient, and thereby enhancing cardiac contractility in the patient. Such methods are effective for the treatment of cardiac disease, cardiac failure, cardiomyopathy, or carditis.

### CROSS REFERENCE TO RELATED APPLICATIONS

This application claims priority to U.S. Provisional Application No. 60/408,398 filed Sep. 4, 2002, which is incorporated herein in its entirety.

### GOVERNMENT INTEREST

This invention was supported in part by Grant No. GM55560 and Independent Scientist Award HL03814 from the National Institutes of Health. Accordingly, the Government may have certain rights in this invention.

### FIELD OF THE INVENTION

The present invention relates to a determination of the relationship between macroscopic conductance of cellular membrane inward-rectifier K⁺ (Kir) ion channels and intrinsic voltage dependence, and to methods for controlling the inward-rectification activity of IRK1 channels and their homologs.

### BACKGROUND OF THE INVENTION

The aqueous compartments within a cell are delineated by cell membranes, consisting primarily of a continuous double layer of lipid molecules associated with various integral or peripheral membrane proteins. Integral membrane proteins include membrane-associated receptors that form pores in the lipid bilayer, gated by ion channel receptors. Ion channel receptors comprise ligand- and voltage-gated channel membrane protein receptors.

Inward-rectifier K⁺ (Kir) channels are a group of membrane proteins that accomplish numerous important physiological tasks, such as regulating cardiac and neuronal electrical activity, coupling insulin secretion to blood glucose level, and maintaining electrolyte balance. Inward rectifiers are so named because they act as bio-diodes in the cell membrane, that selectively conduct K⁺ ions. Namely, they can carry much larger K⁺ currents into a cell at membrane voltages that are more negative than the K⁺ equilibrium potential, but they carry a much smaller current out of a cell at voltages that are more positive than the K⁺ equilibrium potential, even when the K⁺ concentrations on both sides of the membrane are equalized. Consequently, with identical K⁺ concentrations on both sides of the membrane, the steady-state outward macroscopic current at positive membrane voltages is much smaller than the inward current at the corresponding negative voltages. This feature is known as “initially anomalous rectification and subsequently inward rectification” (Katz, Arch. Sci. Physiol. 2:285-299 (1949); Noble, J. Cell. Comp. Physiol. 66:127-136 (1965)).

The diode-like property, i.e., inward rectification, underlies the ability of Kir channels to perform the numerous vital physiological functions. As a result, membrane depolarization-elicited outward current through inward-rectifier potassium (IRK1) channels exhibits profound relaxation. It was originally hypothesized that an action potential inward rectification results in a decrease in potassium conductance upon membrane depolarization, producing the long plateau in the action potential, followed by an increase in potassium conductance during membrane repolarization, which in turn accelerates the latter process (Noble, J. Physiology 160:317-352 (1962)). This predicted that inhibition of inward rectifiers in cardiac ventricular myocytes would prolong the action potential. Lengthening the action potential keeps voltage-gated Ca²⁺ channels open longer, enhancing Ca²⁺ entry into myocytes, and thus cardiac contractility. Early work by Armstrong and Binstock, J Gen. Physiol. 48: 859-872 (1965) further showed that intracellular TEA blocks squid voltage-activated K⁺ channels in a strongly voltage-dependent manner, rendering the channels inwardly rectifying.

Mg²⁺ was later identified as an endogenous voltage-dependent channel blocker that also caused inward rectification (Matsuda et al., Nature 325:156-159 (1987); Vandenberg, Proc. Natl. Acad. Sci. USA 84:2560-2564 (1987)). However, the voltage dependence of the channel block by Mg²⁺ alone was too weak to account for the strong inward rectification observed in intact cells. Furthermore, significant rectification remained in the absence of Mg²⁺. Thus, the concept of intrinsic (voltage-dependent) channel gating was developed to explain Mg²⁺-independent current relaxation and rectification (e.g., Ishihara et al., J. Physiol. 419:287-320 (1989); Silver and DeCoursey, J. Gen. Physiol. 96:109-133 (1990); Stanfield et al., J. Physiol. 475:1-7 (1994)).

Certain polyamines were also found to endogenously block the channels in a strongly voltage-dependent manner (Lopatin et al., Nature 372:366-369 (1994); Ficker et al., Science 266:1068-1072 (1994); Fakler et al., Cell 80:149-154 (1995)). Still, variable residual inward rectification remained after the inside of a membrane patch was perfused with solutions nominally devoid of polyamines and Mg²⁺ (Aleksandrov et al., Biophys. J. 70:2680-2687 (1996); Shieh et al., J. Physiol. 494:363-376 (1996); Lee et al., J. Gen. Physiol. 113:555-565 (1999)). This finding supported the hypothesis that inward rectification results from intrinsic channel gating, modulated by the binding of Mg²⁺ and polyamines to putative channel-gating machinery, rather than from a voltage-dependent channel block by the intracellular cations. However, the laboratory of the present inventor showed that the residual rectification, independent of Mg²⁺ and polyamines, is related to the presence of HEPES (4-(2 hydroxyethyl)-1-piperazineethanesulfonic acid) in the recording solutions (Guo and Lu, J. Gen. Physiol. 116:561-568 (2000a)).

Voltage jump-induced, time-dependent relaxation is not exclusively associated with outward currents, since hyperpolarization-induced inward currents also exhibited relaxation (Kubo et al., Nature 362:127-133 (1993)). Choe et al., as reported in Biophys. J. 78:1988-2003 (1999), showed that hyperpolarization reduced the open probability of IRK1, and argued that the reduction in channel open probability results from both channel blocking by extracellular divalent cations and by channel gating. Similar phenomena occur to a lesser extent in ROMK2 (Choe et al., J. Gen. Physiol. 112:433-446 (1998)). Using IRK1-ROMK2 chimeras, Choe et al. (1999) found that the protein segments that form the ion conduction pore underlie the putative channel gating. Consistent with this finding, T. Lu et al., reported in Nature Neuroscience 4:239-246 (2001) that gating of IRK1 at negative voltages is significant perturbed when ester carbonyls replace the amide carbonyls of the two glycine residues within the signature sequence that forms the ion selectivity filter. Also, Shieh, J. Physiol. 526:241-252 (2000) showed that in low-K⁺ solutions in the absence of extracellular divalent cations, hyperpolarization induces a significant inward current relaxation, which is likened to the C-type inactivation of voltage-activated Shaker K⁺ channels (Hoshi et al., Neuron 7:547-556 (1991); Lopez-Bameo et al., Receptors Channels 1:61-71 (1993); Yellen et al., Biophys. J. 66:1068-1075 (1994)).

Accordingly, there has existed a need to resolve the fundamental issue of whether the macroscopic conductance of IRK1 has any significant intrinsic voltage dependence, and to define the optimal experimental conditions for studying IRK1, as well as for regulating the activity of IRK1 channels and their homologs.

### SUMMARY OF THE INVENTION

In intact cells the depolarization-induced outward IRK1 currents undergo profound relaxation, such that the steady-state macroscopic I-V curve exhibits strong inward rectification. Thus, past researchers have concluded (i) that variable residual inward rectification is a reflection of intrinsic channel gating by intracellular cations (e.g., Mg²⁺ or polyamines), rather than a simple pore block; and (ii) that IRK1 exhibits significant extracellular K⁺-sensitive relaxation of its inward current. However, in the present invention, a systematic experimental investigation of the causes underlying voltage jump-induced current relaxations demonstrates that such current relaxations actually result from impurities in some common constituents of the recording solutions, such as residual hydroxyethylpiperazine in HEPES and ethylenediamine in EDTA. Consequently, inherently, IRK1 channels are essentially ohmic at the macroscopic level, and the voltage jump-induced current relaxations do not reflect IRK1 gating or a significant intrinsic voltage dependence, but rather the unusually high affinity of the IRK1 pore for cations.

Accordingly, the present invention provides methods for regulating IRK1 activity in the cell membrane in a controlled manner, permitting enhanced voltage jump-induced, time-dependent relaxation of the ion channel in the presence of piperizine or ethylenediamine, or derivatives thereof. In the alternative, by controlling the presence of even trace amounts of residual piperizine or ethylenediamine, or derivatives thereof, in the intracellular or extracellular solution, methods are further provided to reduce or block IRK1 ion channels in the cell membrane. In addition, compositions are provided to achieve such regulation of an ion channel of the cell membrane, particularly to effect strong inward rectification and relaxation of IRK1 channels.

When applied in vivo to a patient in need of control of the cellular ion channels, the present invention provides methods for treating such a patient to effect reduced activity or blockage the inward rectifier ion channel, which will allow voltage-gated Ca²⁺ channels open longer, enhancing Ca²⁺ entry into myocytes, and thus enhancing cardiac contractility in the patient.

Furthermore, the present invention identifies optimal experimental conditions for studying IRK1.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE DRAWINGS

The foregoing summary, as well as the following detailed description of the invention, will be better understood when read in conjunction with the appended drawings, although it is not intended that the invention be so limited.

FIGS. 1A-1F provide comparisons of the effects of intracellular HEPES, HEP, and piperazine on IRK1 currents (structures shown at top). FIGS. 1A-1C: Currents were recorded with 10 mM HEPES, 3 μM HEP and 0.3 μM piperazine, respectively, from the same inside-out patch using the described voltage pulse protocol. Intracellular and extracellular solutions were constant, except for the tested chemicals. Currents are corrected for background current. Dotted lines identify the zero current level. FIGS. 1D-1F: Normalized I-V curves, corresponding to FIGS. 1A-1C, constructed from the currents determined at the end of each test voltage-pulse. Current at each voltage is normalized (except for its signs) to that at −100 mV.

FIGS. 2A-2C compare HEPES, HEP and piperazine concentration dependence on the channel block. The fraction of unblocked currents in the presence of three representative concentrations of HEPES (A), HEP(B) or piperazine (C) is plotted against membrane voltage. The theoretical curves are fits of the Woodhull equation, which give K_{d} (0 mV)=2.47±0.02 M (mean±sem, n=6) and Z (valence)=1.02±0.02 for HEPES, K_{d} (0 mV)=2.97±0.16 MM (n=6) and Z=1.09±0.03 for HEP, and K_{d} (0 mV)=0.27±0.04 mM (n=8) and Z=1.08±0.02 for piperazine.

FIGS. 3A-3E shown other causes underlying apparent inward rectification. FIGS. 3A-3D: Currents were recorded using the same inside-out patch with the voltage pulse protocol shown in FIG. 1. The intracellular solution composition (besides KCl) and pH for each panel are indicated. FIG. 3E: Normalized I-V curves constructed from the currents determined at the end of each test voltage-pulse. The I-V curves (a)-(d) correspond to the currents shown in FIGS. 3A-3D, respectively. All data points are mean±sem (n=5).

FIGS. 4A and 4B show IRK1 currents in the presence of three metal ion chelators. FIG. 4A: Current records were collected from the same patch in the presence of intracellular EDTA, EGTA or CDTA, each at 5 mM. In FIG. 4B, normalized I-V curves are depicted in the presence of the metal ion chelators. All data points are mean±sem (n=5).

FIG. 5 depicts the effect of EDTA concentration on IRK1 currents. All traces were recorded from the same patch, with intracellular EDTA concentrations as indicated. The corresponding I-V curves are shown in FIG. 6.

FIGS. 6A-6C show the effects of various EDTA concentrations on the normalized I-V curves of IRK1 channels. For clarity, I-V curves with 0.1 to 5 mM intracellular EDTA (FIG. 6A) are plotted separately from those with 5 to 30 mM EDTA (FIG. 6B). All data points are mean±sem (n=4-6). FIG. 6C graphically shows normalized current at ⁺80 mV, taken from the I-V curves in FIGS. 6A and 6B, plotted against the concentration of EDTA. The data represented by the circles (mean±sem) were determined experimentally, whereas those by triangles were calculated using Equation 1, below.

FIGS. 7A-7D show comparisons of the effect of a channel blocked by EDTA with a block by Mg²⁺ or ethylenediamine (ED). FIGS. 7A and 7C: Currents with 0.1 mM and 30 mM EDTA normalized to that with 5 mM EDTA are plotted against membrane voltage, respectively. FIGS. 7B and 7D: Fraction of unblocked currents in the presence of Mg²⁺ or ED, respectively, is plotted against membrane voltage. All data points are mean±sem (n=5). All curves are fits of the equation I/I₀=1/(1+[blocker]/K_{d}), where K_{d}=K_{d}(0mV)e^{-ZFV/RT}. For FIGS. 7A and 7B, the two nearly superimposed curves for each data set fits either all data points (continuous curves), or all but the rightmost three data points (dashed curves). For FIGS. 7C or 7D, the dashed curve through each data set is a fit to all but the rightmost.3 data points.

FIGS. 8A-8D show comparisons of the effects on IRK1 currents of intracellular EDTA and ethylenediamine (structures shown at top). FIGS. 8A and 8B: Currents were recorded from the same membrane patch with, respectively, 30 mM EDTA and 0.1 μM ethylenediamine. FIGS. 8C and 8D: Normalized I-V curves, wherein each data point represents the mean (±sem; n=5) of currents.

FIG. 9 shows the effects of intracellular pH on the currents of wild-type and D172N mutant IRK1 channels. Currents were recorded at various intracellular pH levels, with extracellular pH=7.6 throughout. For each channel type, all currents were obtained from the same inside-out patch.

FIGS. 10A-10D show the effects of intracellular pH on the I-V curves of wild-type and mutant channels. FIGS. 10A and 10B: I-V curves of wild-type IRK1 and D172N mutant channels at various intracellular pH, determined from the current records, as shown and including those in FIG. 9, except for those at pH 7.6 which were taken from those as shown in FIG. 1A. FIGS. 10C and 10D: Currents through IRK1 and D172N channels normalized to those at pH 8.5, are plotted against membrane voltage. All data points are mean±sem (n=5).

FIGS. 11A and 11B show variable degree and rate of removal of endogenous blockers by perfusion. Current traces shown in FIGS. 11A and 11B were recorded from two separate patches excised from two oocytes injected with different amounts of cRNA (higher in FIG. 11A than in FIG. 11B). The recordings were made at the indicated times following the start of perfusion.

FIGS. 12A-12C show relaxation of inward currents caused by extracellular divalent cations. Currents were recorded with the voltage protocol as shown (0 m V to −200 mV). Intra- and extracellular solutions were buffered with 10 mM phosphate. The extracellular solution contained 0.3 mM Ca²⁺ and 1 mM Mg²⁺ in FIG. 12A, but 5 mM EDTA and no added Ca²⁺ or Mg²⁺ in FIGS. 12B or 12C. The ratio of currents at the end and the beginning of voltage pulses (I_{end}/I_{bgn}) is plotted against membrane voltage. In FIG. 12C, the data corresponding to FIGS. 12A and 12B are labeled by letters (a) and (b), respectively. All data points are mean±sem (n=5).

FIGS. 13A-13D show K⁺ dependence of the inward current relaxation in the presence of extracellular HEPES. All currents were elicited with the voltage protocol used in FIG. 12, using 5 mM EDTA and no added divalent cations in the extracellular solution. Extracellular solutions were buffered with 10 mM. HEPES (FIGS. 13A and 13B) or phosphate (FIGS. 13C and 13D), and contained either 100 mM K⁺ (FIGS. 13A and 13C) or 20 mM K⁺ (FIGS. 13B and 13D). FIG. 13E: Ratio of currents at the end and the beginning of voltage pulses (I_{end}/I_{bgn}) is plotted against membrane voltage. The data corresponding to FIGS. 13A-13D are labeled (a)-(d), respectively. All data points are mean±sem (n 5).

FIGS. 14A-14E show K⁺ dependence of inward current relaxation in the presence of extracellular HEP or piperazine. Intra- and extracellular solutions were buffered with 10 mM phosphate. The extracellular solution contained 3 μM HEP (FIGS. 14A and 14B) or 0.3 μM piperazine (FIGS. 14C and 14D); and either 100 mM K⁺ (FIGS. 14A and 14C) or 20 mM K⁺ (FIGS. 14B and 14D). FIG. 14E: Ratio of currents at the end and the beginning of voltage pulses (I_{end}/I_{bgn}) is plotted against membrane voltage. The data corresponding to panels FIGS. 14A-14D are labeled (a)-(d), respectively. All data points are mean±sem (n=5).

### DETAILED DESCRIPTION OF PREFERRED EMBODIMENTS OF THE INVENTION

In the present invention, a systematically investigation was conducted into the causes underlying the reported IRK1 current relaxations following step changes of membrane voltage, and the findings showed that none of the IRK1 current relaxations is intrinsic to the channels. In fact, contrary to the generally accepted understanding, within the usual voltage range, the macroscopic IRK1 conductance has no significant intrinsic voltage dependence that causes either inward rectification or inactivation. The reported apparent voltage dependence of macroscopic IRK1 currents is instead caused by traces of contaminants in routinely used chemicals, such as HEPES and metal ion chelators. Accordingly, the present invention provides methods for controlling the activity of IRK1 ion channels and homologs thereof, as well as providing the optimal conditions for examining such channels.

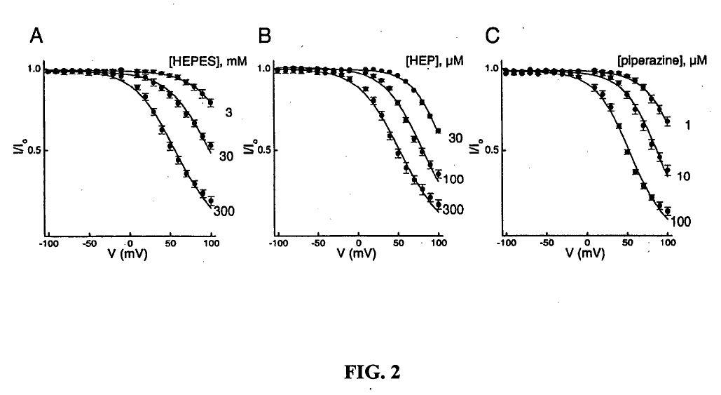

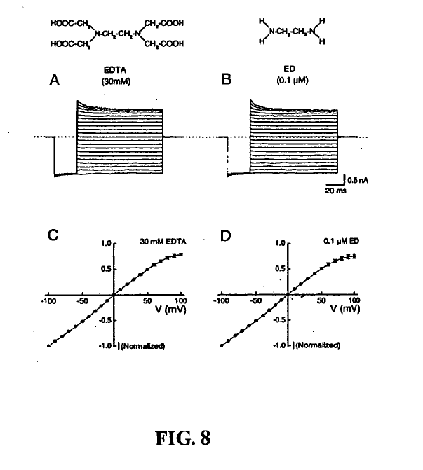

As among the metal ion chelators used as buffers in routine experiments, EDTA (ethylene diamine tetraacetic acid) has the least effect. Specifically, channel blocking effects that have been associated in the literature with both intracellular and extracellular HEPES, have been accounted for in the Examples which follow, by residual HEP (hydroxyethyl piperazine) (˜500 ppm, part per million, in weight). Similarly, as shown below, effects associated with intracellular EDTA are, in fact, caused by residual ED (ethylenediamine) (<5000 ppm). These contamination levels, estimated from the relative specific inhibitory activity of compared chemicals (FIGS. 2 and 8), are well below the limits (<5000 ppm) specified by the supplier.

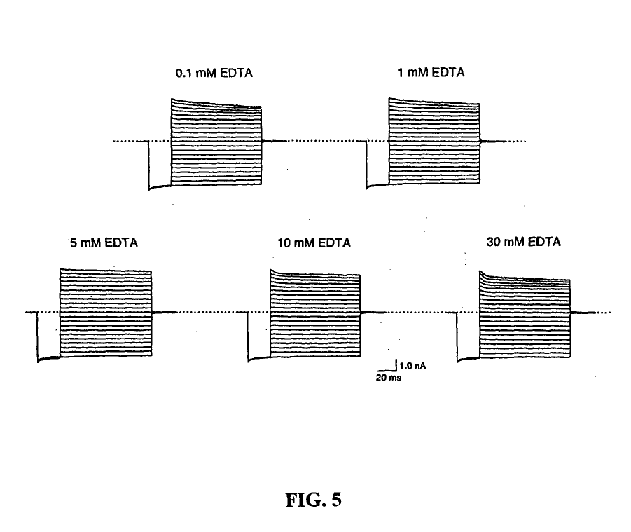

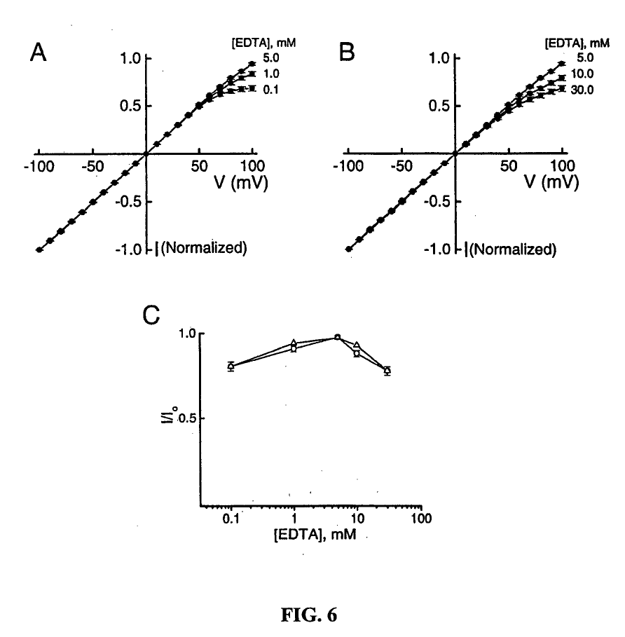

Intracellular divalent chelators must be used both to suppress endogenous Ca²⁺-activated 1⁻ currents and to minimize channel block by contaminating divalent cations. However, a divalent cation chelator, such as EDTA has two opposing effects: 1) it reduces free divalent cation concentration, and thereby relieves channel block by these ions, and 2) it contains residual ethylenediamine that actually blocks the channels. Consequently, the current is a biphasic function of EDTA concentration, having a maximum effect at an intermediate concentration of EDTA (FIGS. 6A-6C). This may vary somewhat, depending on the extent of contamination in a given lot of EDTA, and on the level of divalent cation concentration in the solution. Under the, present solution conditions, using the maximal current corresponding to 5 mM EDTA, there is little time-dependent relaxation of outward current during a 100-msecond test-pulse to positive voltage (even up to ⁺100 mV; FIG. 5), which is sufficiently long for many common studies.

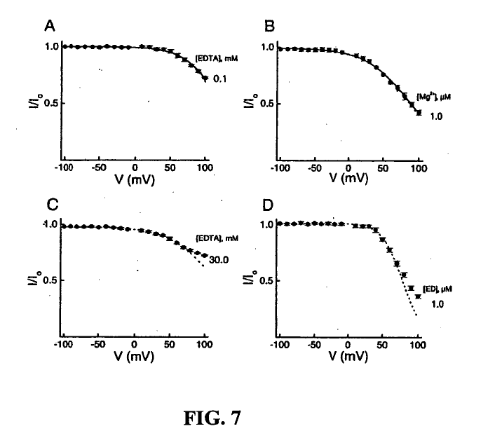

In a solution with 0.1 mM EDTA, the estimated concentration of contaminating ethylenediamine is about 0.3 nM (FIGS. 8A-8D), which causes practically no channel block, given that the ^{ED}K_{d} (0 mV)=1.4 mM and ^{ED}Z=2 (Guo and Lu, 2000b) (ED=ethylenediamine; Z=valence). Therefore, at such a low concentration of EDTA, the channels are blocked primarily by contaminating metal ions (FIGS. 7A-7D). The common divalent cations, Ca²⁺ and Mg²⁺, block inward rectifiers by the same mechanism, although the latter binds with somewhat higher affinity (Matsuda, J. Physiol. 435:83-99 (1993); Matsuda and Cruz, J. Physiol. 470:295-311 (1993)).

Examining the block by intracellular Ca²⁺ is technically more difficult due to the presence of Ca²⁺-activated C1⁻ currents in oocytes, as a result the total concentration of contaminating divalent cations in a preferred embodiment was estimated in terms of the equivalent Mg²⁺. To do so, ^{Mg}K_{d} (0 mV)=17 μM and ^{Mg} Z=1.1 were first determined from the fit of the Woodhull equation to the Mg²⁺-inhibition curve (FIG. 7B). Then, based on these values, it was determined that, from the same analysis of current inhibition in 0.1 mM EDTA, that the extent of channel block is equivalent to that caused by 68 nM free Mg²⁺ (FIG. 7A). The free Mg²⁺ concentration requires 20 μM total Mg²⁺ in a 0.1 mM EDTA solution, while the EDTA-Mg²⁺ stability constant is 3.5×10⁶ M⁻¹. Theoretically, 20 μM Mg²⁺ in a 30 mM EDTA solution results in 22 μM free Mg²⁺, which would cause practically no block. Thus, at high EDTA concentration, the channels are primarily blocked by contaminating ethylenediamine (0.1 μM; FIG. 8).

With these estimates, the current at +80 mV was calculated for each EDTA concentration using the following equation:

$$\frac{I}{{I}_{O}} = \frac{1}{1 + \frac{{[ {Mg}^{2 +} ]}_{free}}{{}_{}^{Mg}{K}{}_{d}^{} ( 0 mV ) {ⅇ}^{- \frac{{}^{Mg} ZFV}{RT}}} + \frac{[ ED ]}{{}_{}^{ED}{K}{}_{d}^{} ( 0 mV ) {ⅇ}^{- \frac{{}^{ED} ZFV}{RT}}}} \qquad Equation 1$$

wherein quantities F (Faraday constant), V (membrane voltage), R (gas constant), and T (Absolute temperature). Since the current at +80 mV does not significantly deviate from the fit of the Woodhull equation (FIGS. 7C and 7D), ethylenediamine is assumed, for simplicity, to be non-permeant. The calculated values based upon the equation agree well with those that were experimentally observed (FIG. 6C).

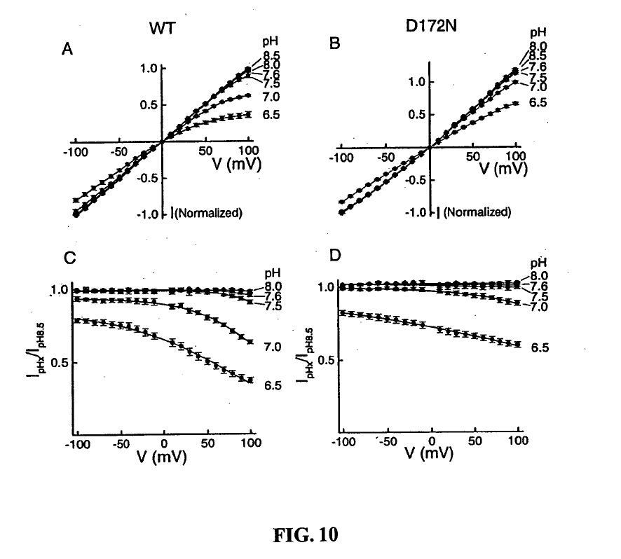

As in the case of other inward rectifiers, lowering intracellular pH significantly inhibits IRK1 (Shieh et al., 1996). FIGS. 10A and 10C show that inhibition by protons has both voltage-dependent and -independent components. Several, residues underlying voltage-independent inhibition by intracellular protons have been identified in various inward rectifiers (e.g., Fakler et al., EMBO J. 16:4093-4099 (1996); Xu et al., Am: J. Cell Physiol. 279:C1464-C1471 (2000)). For example, if amino acid D172, an asparagine in the pore of IRK1, acts as a surface charge, and if protonation of D172 is voltage-dependent, protonation of D172 (changing the charged D⁻ to a neutral D) would theoretically reduce the single channel conductance, and thereby render the channels inwardly rectifying. However, for the reasons set forth herein, this does not appear to be the primary cause underlying the observed voltage-dependent channel inhibition at low pH. Replacing acidic D172 with neutral asparagine (D172N) does not reduce the single channel conductance (Oishi et al., J. Physiol. 510:615-683 (1998). Furthermore, it renders neither the single channel I-V curve, nor the macroscopic I-V curve, inwardly rectifying (Oishi et al., 1998; Guo and Lu, 2000a).

In fact, voltage-dependent inhibition of IRK1 by intracellular protons, probably primarily reflects protonation of amine groups in both the residual endogenous and contaminating exogenous organic blockers and/or EDTA. Protonation of these blockers enhances their affinity for IRK1 (Guo and Lu, 2000b), whereas protonation of EDTA reduces its affinity for trace divalent cations, thus causing further channel blockage. Consistent with this reasoning, mutant D172N channels, whose affinity for intracellular blocking cations is dramatically reduced (Lopatin et al., 1994; Ficker et al., 1994; Fakler et al., 1995; Yang et al., Neuron 14:1047-1054 (1995)), exhibit dramatically reduced voltage-dependent inhibition at low pH (FIG. 10D). In any case, for practical purposes one can obtain essentially uninhibited IRK1 currents at intracellular pH 7.6 or higher.

The affinity of IRK1 channels for some endogenous blockers is exceedingly high, so that even trace amounts of endogenous blocker may cause significant inward rectification. For example, the K_{d} (0 mV) for the binding of fully protonated spermine is ˜10⁻⁷ M, while the effective valence of channel block by spermine is ˜5 (Guo and Lu, 2000a; Guo and Lu 2000b). The calculated K_{d}(+100 mV) for spermine binding is, therefore, ˜10¹⁵ M, which is almost certainly below the concentration of spermine remaining in an exhaustively perfused membrane patch. Therefore, although spermine is a permeant blocker, whose effect can be somewhat relieved by membrane depolarization, at +100 mV in the steady state, most channels will be blocked by spermine at concentrations as low as 1 nM (typical oocyte concentrations are submillimolar; (Osborne et al., Biochem. Biophys. Res. Commun. 158:520-526 (1989))).

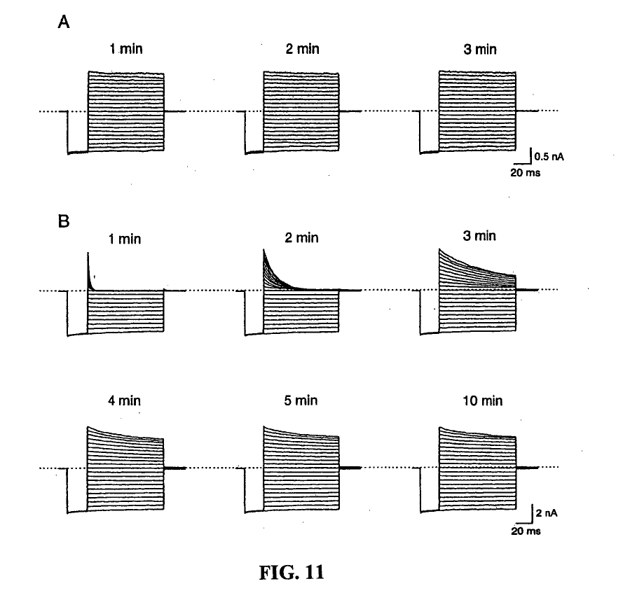

Assuming the rate constant for spermine binding at +100 mV is diffusion-limited and as high as estimated for quaternary ammoniums (10⁸-10⁹ M⁻¹ s⁻¹; Guo and Lu, J. Gen. Physiol. 117:395-405 (2001)), the predicted current reduction caused by 1 nM spermine is between 1% and 10% at the end of a 100 msecond voltage pulse to +100 mV (full steady-state inhibition would require many seconds). Consequently, to limit the extent of channel block by spermine to at most a few percent during a 100-msecond pulse, spermine concentration may need to be reduced to 1 nM or less. Even if the precise values of K_{d} and kₒₙ (at +100 mV) (wherein kₒₙ refers to concentration at which a molecule binds) for spermine are unknown, the above exercise illustrates the practical challenge posed by the need to lower spermine concentration to a level that will leave channel currents essentially unaffected. Not surprisingly, in cases where endogenous blockers cannot be adequately removed despite exhaustive perfusion, significant voltage-dependent channel inhibition will persist (FIGS. 11A; 11B).

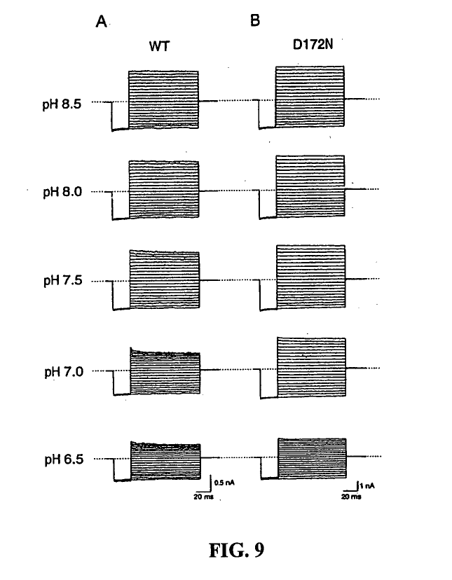

The problem of residual high-affinity inhibitors can be dramatically relieved, or even practically eliminated, by lowering channel affinity for intracellular cations. For example, a linear I-V curve is readily obtained in IRK1 channels containing the D172N mutation (FIGS. 9A-9B; 10A-10D; Guo and Lu, 2000a), which significantly lowers their affinity for intracellular spermine (e.g., Yang et al., 1995). Also as expected, satisfactory removal of endogenous blockers was possible only with very small patches (FIGS. 11A-11B). As reported in Example 5 below, to more effectively perfuse the patch, the tip of the patch pipette was positioned in a rapid stream of the intracellular solution, as opposed to perfusing the entire recording chamber. In addition, the oocytes were kept away from the recorded patch, since oocytes are known to release substantial amounts of polyamines (Lopatin et al., 1994; Ficker et al., 1994).

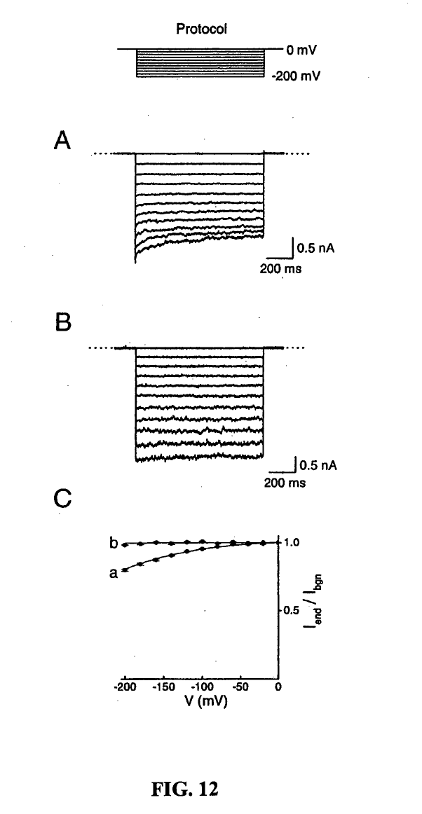

As noted above, relaxation of inward IRK1 current induced by hyperpolarization has also been observed. Choe et al., (1999) found, as confirmed in Example 6 below (FIG. 12A-12C), that some inward current relaxation results from channel block by divalent cations in the extracellular solution. Furthermore, Shieh (2000) showed, in the absence of extracellular divalent cations, but in the presence of HEPES, that lowering K⁺ concentration reveals profound current relaxation following strong membrane hyperpolarization. Based on this finding, the it was suggested that the current relaxation resembles C-type inactivation of Shaker voltage-activated K⁺ channels, which is similarly “protected” by K⁺.

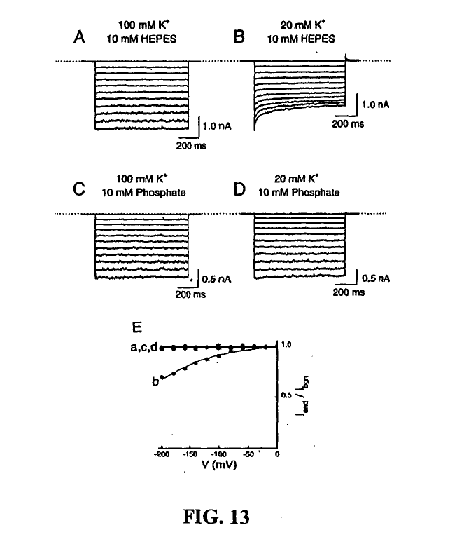

However, preferred embodiments of the present invention show that the K⁺-sensitive inward current relaxation can be also accounted for by residual HEP in the HEPES used to buffer extracellular pH (FIGS. 13A-13E; 14A-14E). On that basis, it is now evident that the K⁺-sensitive current relaxation is not an intrinsic gating property of these channels. The dramatic channel block induced by lowering K⁺ on both sides of the membrane in the art, probably resulted from both a reduced competition of extracellular K⁺ with extracellular blocking ions and a reduced “knock off” effect of the blocking ions by intracellular K⁺ (Armstrong and Binstock, 1965; Armstrong, J. Gen. Physiol. 58:413-437 (1971); Yellen, J. Gen. Physiol. 84:187-199 (1984); Neyton and Miller, J. Gen. Physiol. 92:549-567 (1988a); Neyton and Miller, J. Gen. Physiol. 92:569-586 (1988b); MacKinnon and Miller, J. Gen. Physiol. 91:335-349 (1988); Spassova and Lu, J. Gen. Physiol. 112:211-221 (1998)).

Taken together, at the macroscopic level IRK1 channels inherently have practically ohmic characteristics, although in principle the I-V curve may exhibit very slight outward rectification in the complete absence of any endogenous or exogenous blockers. In intact cells, the observed inward rectification of the I-V curve results from voltage-dependent block by intracellular cations such as Mg²⁺ and polyamines (Matsuda et al., 1987; Vandenberg, 1987; Ficker et al., 1994; Lopatin et al., 1994; Fakler et al., 1995). However, in excised membrane patches, perfused with solutions nominally devoid of Mg²⁺ and polyamines, the relaxation of both inward and outward IRK1 currents induced by voltage jumps and the resulting non-linearity of the I-V curve is a reflection, not of intrinsic gating properties of IRK1 channels, but of the unusually high affinity of IRK1 for cations. Because of the extraordinarily high affinity for cations, traces of contaminants in commonly used organic pH buffers and metal ion chelators become highly significant and problematic in the study of IRK1 channels, even though prior to the present invention such trace levels of contaminants would usually have been considered insignificant. Despite this, practically uninhibited (inward and outward) IRK1 currents, and therefore linear I-V curves, can be obtained, provided that the recorded membrane patch is adequately perfused and that both intracellular and extracellular solutions contain 100 mM KCI, 5 mM EDTA and 10 mM phosphate at pH 7.6 or above.

Accordingly in either in vivo cell membranes or in vitro studies using membrane patches, it is possible in accordance with preferred embodiments of the present invention to control and/or regulate the activity of a Kir ion channel, in particular of an IRK1 channel. Activity (flow of current) can be either enhanced or reduced.

Moreover, embodiments of the present invention (for instance, as set forth in the Examples that follow) provide methods examine the activity of the channel without significant interference by contaminants in either intracellular or extracellular solution.

In Vivo Methods of Controlling Activity of Inward-Rectifier K⁺ Ion Channels

Recent studies have shown that Kir2.1 is the predominant inward rectifier K⁺ channel in cardiac ventricular myocytes (Brahmajothi et al., Circulation Research 78:1083-1089 (1996); Nakamura et al., Amer. J Physiol. 274: H892-900 (1998); Zaritsky et al., J. Physiol. 533:697-710 (2001)). By genetically knocking out the gene encoding Kir2.1 in mice, it was seen that the action potential was prolonged, as was the Q-T interval in EKG. However, Kir2.1^{-/-} mice show no sign of any significant cardiac arrhythmia (Zaritsky et al., 2001). Studies from the present inventor's laboratory have shown that piperazine (and its derivatives) inhibits Kir 2.1 from the intracellular side. (Guo and Lu, J. Gen. Physiol. 120:539-551 (2002)). Piperazine inhibition of Kir2.1 appears to be specific, since piperazine at 100 μM dramatically inhibits Kir2. 1, but has no effects on a homologous inward rectifier (Kir1.1), or on a voltage-gated K⁺ channel (the Shaker channel). This apparent specificity results from the presence of two acidic residues (E224 and E299) in Kir2.1. By replacing these two residues with neutral ones rendered the channel practically insensitive to 100 μM piperazine.

Thus, in light of the foregoing findings, piperazine or its derivatives may be used to increase cardiac contractility to treat certain cardiac diseases, such as cardiac failure, cardiomyopathy, carditis, or the like in a patient. Since repolarization of the action potential is mainly caused, not by outward K⁺ currents through Kir channels, but those through voltage-gated K⁺ channels (including HERG), modest inhibition of Kir channels should not lengthen the action potential so much that it causes the long QT syndrome.

According to Goodman & Gilman's, The Pharmacological Basis of Therapeutics, McGraw-Hill, New York (1996), piperazine citrate (vermizine), the form of the drug available in the United States, is useful and inexpensive second-choice alternative to mebendazole or pyrantel pamoate in treating combined ascariasis and enterobius infections. Accordingly, it is known to be safe for the treatment of humans under recognized conditions and dosages. Piperazine preparations (tables or syrup) are typically given orally. For example, in ascariasis, accepted therapy is to give 75 mg/kg, to a maximum of 3.5 grams, as a single daily does for 2 consecutive days. Children should be treated in the same way. Piperazine has been used without ill effect during pregnancy. There is a significant difference between effective therapeutic and overtly toxic doses of piperazine. Thus, there is little danger of overdose using the drug. Laboratory studies on patients receiving treatment for several days have shown no abnormality. Occasional gastrointestinal upset, transient neurological effects, and urticarial reaction have attended its use. Piperazine has pKₐ of ˜4 and is essentially deprotonated in a solution with near neutral pH.

Thus, piperazine is membrane permeable, making it possible to infuse piperazine, or derivatives thereof, into the cells to control activity of the ion channel as a method of treating patients in need of enhancement of cardiac contractility by prolonging the cardiac action potential, which causes the voltage-gated Ca²⁺ channels to stay open longer than they would be open absent such addition of piperazine; thereby enhancing Ca²⁺ entry into myocytes of the patient, and thereby enhancing cardiac contractility in the patient.

The following examples illustrate certain preferred modes of making and practicing the present invention, but are not meant to limit the scope of the invention since alternative methods may be utilized to obtain similar results.

### EXAMPLES

The following materials and methods were used in the Examples that follow:

Molecular Biology and Oocyte Preparation

IRK1 cDNA (Kubo et al., 1993) was cloned into the pGEM-Hess plasmid (IRK1-pGEMHess construct, provided by J. Yang). RNA was synthesized using T7 polymerase (Promega Corp., Madison, Wis.) from Nhe1-linearized cDNAs. Oocytes harvested from Xenopus laevis (Xenopus One, Ann Arbor, Mich.) were incubated in a solution containing NaCl, 82.5 mM; KCI, 2.5 mM; MgCl₂, 1.0 mM; HEPES (pH 7.6), 5.0 mM and collagenase, 2-4 mg/ml. The oocyte preparation was agitated at 80 rpm for 60-90 minutes. It was then rinsed thoroughly and stored in a solution containing NaCl, 96 mM; KCl, 2.5 mM; CaCl₂, 1.8 mM; MgCl₂, 1.0 mM; HEPES (pH 7.6), 5 mM and gentamicin, 50 μg/ml. Defolliculated oocytes were selected and injected with RNA at least 2 and 16 hours, respectively, after collagenase treatment. All oocytes were stored at 18° C.

Patch Recording

IRK1 currents were recorded from inside-out membrane patches of Xenopus oocytes (injected with IRK1cRNA) with an Axopatch 200B amplifier (Axon Instruments, Inc., Foster City, Calif.), filtered at 5 kHz, and sampled at 25 kHz using an analog-to-digital converter (DigiData 1200, Axon Instruments) interfaced with a personal computer. pClamp6 software (Axon Instruments) was used to control the amplifier and acquire the data. During current recording, the voltage across the membrane patch was first hyperpolarized from the 0 mV holding potential to −100 mV, and then stepped to various test voltages, or stepped directly from the holding potential. The duration of the voltage test pulse was 100 mseconds, which is comparable to those used in the studies where the Mg²⁺ and polyamine-independent rectification was initially observed (Aleksandrov et al., 1996; Shieh et al., 1996). Background leak current correction was carried out as previously described (Lu and MacKinnon, Nature 371:243-246 (1994); Guo and Lu, 2000a). To effectively perfuse the patch, the tip of the patch pipette (3 MΣ) was immersed in a stream of intracellular solution exiting one of ten glass capillaries (ID=0.2 mm) mounted in parallel.

Recording Solutions

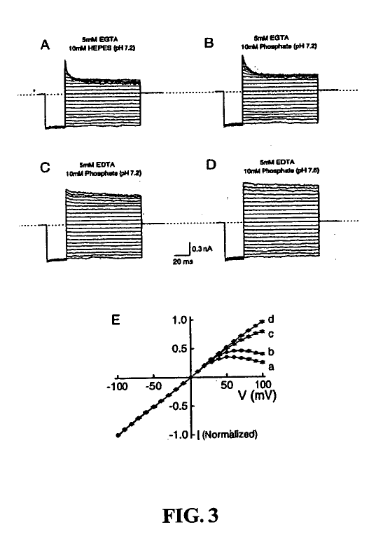

All recording solutions contained 100 mM K⁺ contributed by KCl, K₂EDTA, K₂HPO₄, KH₂PO₄, and KOH. The phosphate-buffered intracellular solution contained 5 mM K₂EDTA (unless specified otherwise), and 10 mM “K₂HPO₄+KH₂PO₄” in a ratio yielding the desired pH, and sufficient KCl to bring total K⁺ concentration to 100 mM (Guo and Lu, 2000a). The HEPES-buffered intracellular solution, as used in FIG. 3, contained (100 mM K⁺ (Cl⁻+OH⁻), 5 mM EDTA and 10 mM HEPES, pH 7.2 (adjusted with KOH). The HEPES-buffered extracellular solution contained 100 mM K⁺ (Cl⁻+OH⁻), 0.3 mM CaCl₂, 1.0 mM MgCl₂, and 10 mM HEPES, pH 7.6 (adjusted with KOH). In the phosphate-buffered extracellular solution, HEPES was replaced by an equal concentration of “K₂HPO₄+KH₂PO₄” in a ratio yielding pH 7.6. The divalent cation-free extracellular solution contained 5 mM EDTA. All chemicals were purchased from Fluka Chemical Corp.

### Example 1

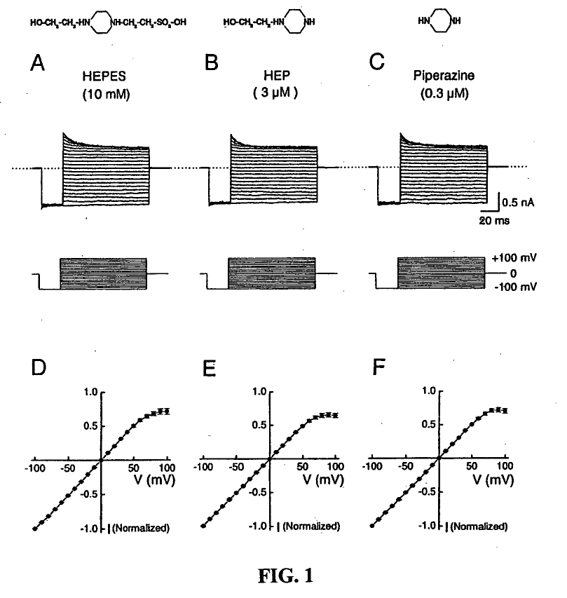

The inventor has previously showed that intracellular “HEPES” blocks IRK1 channels with varying potency depending on the commercial source of the reagent (Guo and Lu, 2000a). To confirm that finding and compare the effects of intracellular HEPES, HEP, and piperazine on IRK1 currents, currents were recorded with 10 mM HEPES, 3 μM HEP and 0.3 μM piperazine, respectively, from the same inside-out patch (prepared as described) using the voltage pulse patch recording protocol. See FIGS. 1A-1C. In all cases the intracellular solutions contained 100 mM K⁺, 5 mM EDTA and 10 mM phosphate (pH 7.6) in addition to the tested chemicals, while the extracellular solution contained 100 mM K⁺, 0.3 mM Ca²⁺, 1 mM Mg²⁺, 10 mM phosphate (pH 7.6). The currents were corrected for background current.

Usually HEPES is synthesized by reacting hydroxyethylpiperazine (HEP) with bromoethanesulfonate (Good et al., Biochemistry 5:467-477 (1966)). As shown in FIG. 1A, the blocking kinetics are slow, even with 10 mM HEPES present. These findings indicated that the block was caused primarily by some impurity in the HEPES. FIG. 1B shows the current records in the presence of 3 μM HEP.

Normalized I-V curves, corresponding to FIGS. 1A-1C, which were constructed from the currents determined at the end of each test voltage-pulse are shown in FIGS. 1D-1F. Current at each voltage was normalized (except for its signs) to that at −100 mV. Each data point in FIGS. 1D-1F represents the mean (sem) of currents recorded from 5-7 patches. The I-V curves determined at the end of the voltage pulses in the presence of 10 mM HEPES and 3 μM HEP are shown in FIGS. 1D and 1E, respectively.

As shown, HEPES and HEP blocked the channels in a qualitatively comparable manner, although HEP was much more potent. Therefore, the blocking activity of HEP must come from the piperazine ring, since piperazine itself is even more potent (FIG. 1B versus 1C, and 1E versus 1F).

### Example 2

FIG. 2 shows the fraction of unblocked currents in the presence of three representative concentrations of HEPES (FIG. 2A), HEP (FIG. 2B), or piperazine. (FIG. 2C) plotted against membrane voltage. The theoretical curves in FIG. 2 were fits with the Woodhull equation, providing an apparent K_{d}(0 mV)=2.47±0.02 M (mean±sem, n=6) and Z (valence)=1.02=1.02+0.02 for HEPES, K_{d} (0 mV)=2.97±0.16 MM (n=6) and Z=1.09±0.03 for and K_{d} (0 mV)=0.27±0.04 mM (n=8) and Z=1.08±0.02 for piperazine. Therefore, the apparent channel block by HEPES can be accounted for by the presence of a trace amount of HEP, which is well below the limit of impurity specified by the supplier (0.5%).

### Example 3

Surprisingly, HEPES is not the sole source of contaminating blockers in commonly used intracellular solutions. Other causes were found to underlie apparent inward rectification. IRK1 channels exhibit inward rectification in the presence of an intracellular solution like that which was used by Aleksandrov et al. (1996), which contained KCI, EGTA (ethylene glycol bis(β-aminoethyl ether) N,N,N′,N′-tetraacetic acid) and HEPES (pH 7.2). Using the same inside-out patch with the voltage pulse protocol shown in FIG. 1, currents were recorded as shown in FIGS. 3A-3D (the intracellular solution composition (besides KCl) and pH for each panel are indicated). The rectification was reduced only modestly when HEPES (10 mM) was replaced by phosphate (10 mM). But as shown in FIG. 3, it was reduced significantly further when EDTA (5 mM) was substituted for EGTA (5 mM), although the normalized I-V curve did not approach linearity until the pH of the solution was raised from 7.2 to 7.6 (FIG. 3E). [The final composition is the one reported by Guo and Lu, 2000a]. The normalized I-V curves were constructed from the currents determined at the end of each test voltage-pulse. All data points were mean±sem (n=5).

These findings show that the persisting rectification after removal of endogenous blockers (Aleksandrov et al., 1996) was caused by the use of HEPES, EGTA, and low pH. Intracellular EGTA, HEPES (each up to 10 mM) and pH<7.4 have been widely used in studies of IRK1.

### Example 4

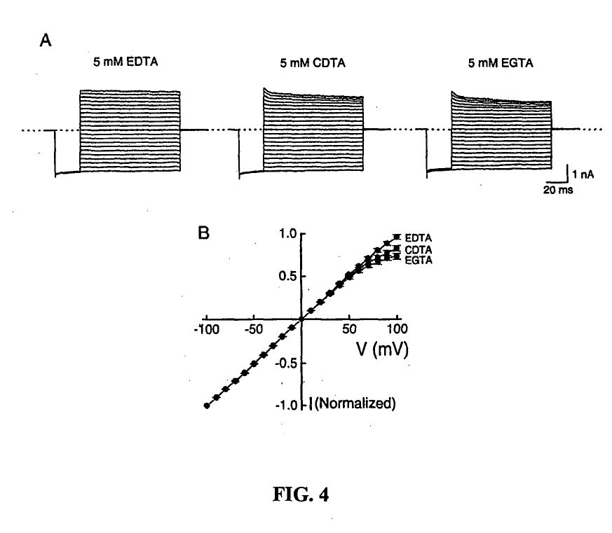

FIG. 4A compares the effects on IRK1 currents in the presence of intracellular metal ion chelators—EDTA, CDTA (cyclohexane 1,2-diaminetetraacetic acid), or EGTA (each at 5 mM; pH 7.6). Phosphate was used to buffer pH. FIG. 4B shows the effect of the same intracellular metal ion chelators by normalized I-V curves. All data points are mean±sem (n=5).

Inward rectification was more pronounced with either EGTA or CDTA, as compared with EDTA, even though the affinity of CDTA for a given di- or trivalent cation is much higher than that of EDTA. Since EDTA causes the least rectification and also is non-selective among divalent cations, EDTA, instead of EGTA, should be used to scavenge contaminating blocking metal ions.

To find the intracellular EDTA concentration that yielded the most linear I-V curve (at pH 7.6), current was recorded from the same patch in the presence of five different concentrations of EDTA (FIG. 5). The corresponding I-V curves are shown in FIG. 6. Solution pH was buffered with phosphate. At 0.1 mM EDTA, outward currents displayed modest inward rectification. As the chelator concentration was raised to 5 mM, the rectification nearly vanished, most probably because the concentration of contaminating free blocking metal ions was reduced. However, when EDTA concentration was increased further, rectification again became more pronounced, presumably reflecting the effect of some impurity in the EDTA.

For clarity I-V curves for 0.1 to 5 mM and for 5 to 30 mM EDTA were plotted separately (FIGS. 6A and 6B), showing that 5 mM EDTA is optimal for obtaining a linear macroscopic I-V curve. All data points were mean±sem (n=4-6). FIG. 6C is a chart showing the normalized current (mean±sem) at ⁺80 mV, taken from the I-V curves in FIGS. 6A and 6B, plotted against the concentration of EDTA that was experimentally determined (circles, FIG. 6C). By comparison, the data represented in FIG. 6C by triangles were calculated using Equation 1.

The fraction of unblocked currents in an IRK1 channel blocked by a low (0.1 mM; FIG. 7A) or a high (30 mM; FIG. 7C) concentration of EDTA was plotted against membrane voltage. The two blocking curves differed in character. At 0.1 mM EDTA, the curve in FIG. 7A was well described by the Woodhull equation (Woodhull, J. Gen. Physiol. 61:687-708 (1973)). Consistent with that finding, the channels were blocked by a “non-permeant” blocker, such as a divalent cation, and plotted against membrane voltage. In FIG. 7B, the block was by Mg²⁺.

In contrast, at 30 mM EDTA the blocking curve deviated from the Woodhull equation (FIG. 7C). Consistent with that finding, the channels were blocked by a permeant blocker, such as ethylenediamine (ED, FIG. 7D) and plotted against membrane voltage. See Guo and Lu, J. Gen. Physiol. 115:799-813 (2000b) for details. All data points were mean±sem (n=5). All curves are fits of the equation I/I₀=1/(1+[blocker]/K_{d}), where K_{d}=K_{d}(0 mV)e^{-ZFV/RT}.

FIGS. 8A and 8B show the effect on IRK1 current records when intracellular 30 mM EDTA and 0.1 μM ethylenediamine, respectively, were used. Currents were recorded from the same membrane patch. The corresponding normalized I-V curves are shown in FIGS, 8C and 8D, wherein each data point represents the mean (±sem; n=5) of currents. EDTA and ethylenediamine blocked the channels in a qualitatively comparable manner, although the latter is more than ten-thousand-fold (10,000×) more potent. Therefore, the blocking effect associated with EDTA can be accounted for by a mere trace of contaminating ethylenediamine used to synthesize the EDTA. Consequently, all subsequent data were recorded with intracellular solutions containing 5 mM EDTA and 10 mM phosphate (without HEPES).

### Example 5

To determine the optimal intracellular pH for recording current activity, an analysis was conducted on the currents of wild-type and D172N mutant IRK1 to examined how varying intracellular pH affects IRK1 currents (FIG. 9A). Currents were recorded at various intracellular pH levels, while extracellular pH was maintained at 7.6 throughout. For each channel type, all currents were obtained from the same inside-out patch. The average I-V curve for each pH examined was plotted in FIG. 10A. As shown, the normalized I-V curve was linear at pH 8.5. Lowering pH inhibited IRK1 currents minimally between pH 8.5 and 7.5, but the change was dramatic between pH 7.5 and 6.5.

The fraction of current not inhibited by protons was plotted in FIG. 10C, which shows that channel inhibition has both voltage-dependent and -independent components. Interestingly, much of the voltage-dependent component of proton inhibition vanished when a mutant channel was used, wherein acidic amino acid 172 (D; located in the inner pore) was replaced by neutral asparagine, referred to as D172N. Currents through the wild-type IRK1 and the D172N IRK1 channels were normalized to those at pH 8.5, and plotted against membrane voltage (FIGS. 9B, 10B and 10D). At higher intracellular pH, the outward D172N current is slightly larger than the inward current. All data points were mean±sem (n=5).

As a practical matter, very small membrane patches must be used to achieve adequate removal (by degree and by rate) of endogenous blockers by perfusion. To illustrate this, FIG. 11A shows three consecutive sets of current records from a small membrane patch, taken at one-minute intervals after the start of perfusion. Any endogenous blockers present in that excised patch were apparently effectively removed within the first minute. In contrast, for a much larger patch (from a separate oocyte injected with much less cRNA), removal of endogenous blockers was very slow and incomplete even after prolonged perfusion (FIG. 11B).

### Example 6

Under certain commonly used experimental conditions membrane hyperpolarization also induces relaxation of inward IRK1 currents. The currents in FIG. 12A (and all of those recorded above) were recorded with 0.3 mM Ca²⁺ and 1.0 mM Mg²⁺ present in the extracellular solution, buffered with phosphate (10 mM). The inward currents at very negative voltages relaxed noticeably. This relaxation largely vanished when 5 mM EDTA was used in the absence of extracellular divalent cations (no added Ca²⁺ or Mg²⁺) (FIG. 12B). (Note that 5 mM EDTA was shown to be optimal for obtaining a linear macroscopic I-V curve in Example 4, above). The ratio of currents at the end as compared with the beginning of the voltage pulses (I_{end}/I_{bgn}) was plotted against membrane voltage (FIG. 12C). All data points were mean±sem (n=5). These results are consistent with the previous finding by Choe et al. (1999) that extracellular divalent cations block IRK1 in a voltage-dependent manner.

Furthermore, Shieh (2000) observed relaxation of inward current even in the nominal absence of extracellular divalent cations. The relaxation reported in that study was prominent only at low concentrations of K⁺. Since solutions in the quoted study were HEPES buffered, currents elicited with the voltage protocol used to produce the data in FIG. 12 were evaluated with no added divalent cations in the extracellular solution to determine whether the current relaxation was due to the use of extracellular HEPES. Indeed, confirming Shieh's (2000) results, the inward current following strong membrane hyperpolarization exhibited little or no relaxation when the extracellular solution contained 100 mM K⁺, 10 mm HEPES and 5 mM EDTA, but no added divalent cations (FIG. 13A). Replacing the 10 mM HEPES buffer in the extracellular solutions with phosphate had no noticeable effect (FIG. 13C).

Further explaining Shieh's finding, maintaining the 10 mM. HEPES buffer in the extracellular solution, but lowering the K⁺ concentration to 20 mM, produced prominent inward current relaxation (FIG. 13B). However, this relaxation vanished when HEPES was replaced with 20 mM phosphate (FIG. 13D). The voltage dependence of the ratio of currents at the beginning and the end of the voltage pulses (I_{end}/I_{bgn}) plotted against membrane voltage is shown in FIG. 13E. All data points were mean±sem (n 5).

### Example 7

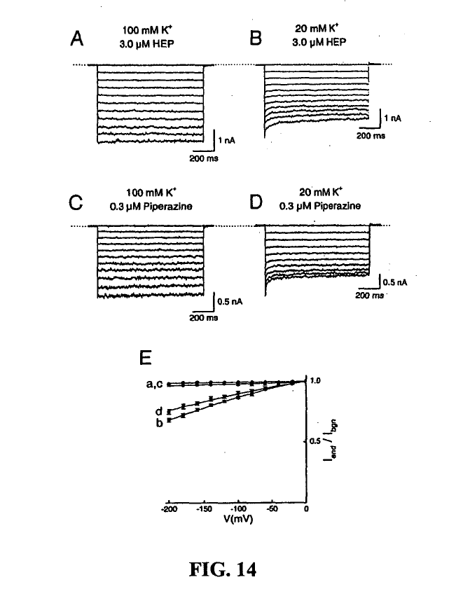

The channel block associated with extracellular HEPES was examined to determine whether it could also be caused by residual contaminants. Intra- and extracellular solutions were buffered with 10 mM phosphate. The extracellular solution contained 3 μM HEP (FIGS. 14A and 14B) or 0.3 μM piperazine (FIGS. 14C and 14D); and either 100 mM K⁺ (FIGS. 14A and 14C) or 20 mM K⁺ (FIGS. 14B and 14D). The ratio of currents at the end and the beginning of voltage pulses (I_{end}/I_{bgn}) was plotted against membrane voltage. All data points were mean±sem (n=5).

As seen in FIG. 14, HEP and piperazine-like “HEPES” blocked the channels in the presence of 20 mM extracellular K⁺, but not 100 mM extracellular K⁺. Therefore, the channel block associated with extracellular HEPES can also be accounted for by a trace amount of contaminating HEP.

The disclosures of each patent, patent application and publication cited or described in this document are hereby incorporated herein by reference, in their entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

I claim:

1. A method for controlling activity of an inward rectifier K⁺ (Kir) ion channel in a cell or cell membrane by controlling an amount of one or more chemical contaminant(s) of an organic pH buffer or metal chelator in intracellular solution.

2. The method of claim 1, wherein the Kir ion channel is IRK1 or a homolog thereof.

3. The method of claim 1, further comprising reducing or blocking activity of the ion channel by adding to the intracellular solution a sufficient amount of the one or more chemical(s) otherwise found as a contaminant of the organic pH buffer or metal chelator to reduce or block current of the ion channel.

4. The method of claim 1, further comprising enhancing activity of the ion channel by removing from the intracellular solution a sufficient amount of the one or more chemical(s) otherwise found as a contaminant of the organic pH buffer or metal chelator to enhance current of the ion channel.

5. The method of claim 1, wherein the metal chelator is selected from the group consisting of EDTA, EGTA, and CDTA.

6. The method of claim 3, wherein the added chemical is piperazine, or a derivative thereof.

7. The method of claim 4, wherein the removed chemical is piperazine, or a derivative thereof.

8. The method of claim 5, wherein a constituent of EDTA, EGTA or CDTA is ethylenediamine, or a derivative thereof.

9. The method of claim 1, wherein the method is applied to the cell or cell membrane in vivo.

10. The method of claim 1, wherein the method is applied to the cell or cell membrane in vitro.

11. A method of treating a patient, wherein the patient requires enhancement of cardiac contractility, said method comprising:
administering to the patient an amount of piperazine, or a derivative thereof, sufficient to reduce or block the activity of at least one inward rectifier ion channel, thereby
prolonging cardiac action potential, which causes voltage-gated Ca²⁺ channels to remain open for a period longer than the voltage-gated Ca²⁺ channels would be open absent such addition of piperazine; thereby
enhancing Ca²⁺ entry into myocytes of the patient, and thereby enhancing cardiac contractility in the patient.

12. The method of claim 11, further comprising treating cardiac disease, cardiac failure, cardiomyopathy, or carditis in the patient.
